(19) United States
(12) Patent Application Publication
(10) Pub. No.: US 2011/0238148 A1
(43) Pub. Date: Sep. 29, 2011

(54) IMPLANT REMOVAL

(76) Inventors: Hermann Monstadt, Bochum (DE); Hans Henkes, Stuttgart (DE); Jörg Ascherfeld, Hattingen (DE); Ralf Hannes, Dortmund (DE)

(21) Appl. No.: 13/122,526

(22) PCT Filed: Oct. 6, 2009
(86) PCT No.: PCT/EP2009/007154

§ 371 Date: Jun. 20, 2011

(30) Foreign Application Priority Data
Oct. 6, 2008 (DE) 10 2008 050 085.2

(51) Int. Cl. A61F 2/84

(52) U.S. Cl. 623/1.11

(57) ABSTRACT

The invention relates to a device comprising an endovascular implant (1) to be introduced into blood vessels or body cavities of the human or animal body and an insertion aid (2), wherein the implant (1) and the insertion aid (2) being connected to each other via a detachment element (3) which is suitable to be dissolved at least partially after the device has been inserted into the body and in this manner causes the implant (1) to become detached or separated from the insertion aid (2), with said detachment element (3) being made of magnesium, a to magnesium alloy and/or a biodegradable material. Such a device enables particularly short detachment times to be attained.

The invention relates to a device comprising an endovascular implant to be introduced into blood vessels or body cavities of the human or animal body and an insertion aid, wherein the implant and the insertion aid being connected to each other via a detachment element which is designed to be dissolved after the device has been inserted into the body and in this manner causes the implant to become detached or separated from the insertion aid.

The use of endovascular techniques for the occlusion of body cavities or vessels such as arteries, veins, fallopian tubes or vascular deformities (for example, vascular aneurysms) is known in the art. For this purpose, so-called occlusion helixes, for example, are introduced by means of an endovascular guide wire serving as insertion aid through a catheter into the cavity to be occluded and deposited therein. Also known is the placement of other implants, for example stents.

To detach the implant from the insertion aid various methods are known from prior art. Aside from mechanical methods the electrolytic severance of stainless steel wire ends as initially described for electro-coagulation by Thompson et al. as well as McAlister et al. in 1979 has in particular proved its worth in this context (Radiology 133:335-340, Nov. 1979; AJR 132:998-1000, June 1979). Based on this, also publication EP 0 484 468 B1 has proposed a device for the implantation of occlusion helixes involving an electrolytic detachment method.

Irrespective of the type of the implant to be inserted at a given time the attending physician always endeavors to keep the detachment time as short as possible to make sure an implant displacement or other undesirable events cannot occur during detachment. For this reason various materials were used in the past for the detachment element arranged between the implant and the insertion aid with a view to enabling said element to be quickly dissolved as soon as a voltage is applied. For example WO 03/017852 A1 has proposed using a stainless steel material which was first subjected to a pre-corrosion process involving a heat treatment.

Another prerequisite linked with the detachment element is that no substances detrimental to human health must develop during the dissolution of the detachment element.

It is therefore the objective of the present invention to propose based on a device of the kind first mentioned above means that on the one hand are suitable to further bring down the time span required to detach the implant from the insertion aid and on the other hand do not exert harmful effects on human health during dissolution.

As proposed by the invention this objective is reached by providing a device comprising an endovascular implant to be introduced into blood vessels or body cavities of the human or animal body and an insertion aid, wherein the implant and the insertion aid being connected to each other via a detachment element which is suitable to be dissolved at least partially after the device has been inserted into the body and in this manner causes the implant to become detached or separated from insertion aid, with the detachment element being made of magnesium, a magnesium alloy and/or a biodegradable material.

Surprisingly, it has been found that the use of magnesium or magnesium alloys for the detachment element offers significant advantages. From initial tests it can be said that when applying a voltage to pure magnesium (Mg 99.9% w/w) detachment takes place within a time span of approx. 5 s and when using magnesium alloys (in this case magnesium—4% w/w yttrium—3% w/w rare earth metals; trade name WE 43) detachment occurs within approx. 15 s which is to be considered a major improvement in comparison to what prior art provides when, for example, publication WO 2005/070308 A2 refers to 20′ to 40 s (2 V, 2 mA). Moreover, through the dissolution of magnesium or magnesium alloys no substances harmful to human health will be produced because magnesium is anyway an element essentially required by the body.

Rare earth metals in this context are scandium, yttrium as well as the lanthanides lanthanum, cerium, praseodymium, neodymium, promethium, samarium, europium, gadolinium, terbium, dysprosium, holmium, erbium, thulium, ytterbium and lutetium. In particular, magnesium alloys containing yttrium and/or rare earth metals can be employed. Advantageously, the contents amount up to 15% w/w, preferably up to 10% w/w. Especially preferred is a magnesium alloy that contains 3.7 to 4.3% w/w of yttrium, 2.4 to 4.4% w/w of further rare earth metals and, if thought expedient, minor amounts of zirconium. Preferred is a content of at least 0.4% w/w of zirconium. Such a magnesium alloy is known by the name WE43.

Aside from magnesium or magnesium alloys other biodegradable materials may also be employed, said materials as a rule being metals or metal alloys as well. For example, pure iron or iron alloys, with the exception of stainless steel, may be used. In this case as well it is beneficial that the substances can be processed by the biological system of the body without difficulty and may then be expediently discharged so that no detrimental side effects are to be expected.

The insertion aid is preferably a customary guide wire of proven design for the purpose of passing occlusion helixes or stents through a catheter to the relevant placement site. The insertion aid is connected to the proximal end of the implant, i.e. in the direction from where the physician pushes the implant forward.

The implant may be an occlusion helix or a stent. Basically however, the invention can be used for any kind of endovascular implant which is to be transferred to its destination by means of an insertion aid from which the implant is then to be disconnected. Implants under scope of the invention are also such objects that are designed for optional detachment only and may either remain in the body or be removed from it depending on relevant treatment necessities. Examples in this respect are retrieval systems designed for the removal of thrombi.

Advantageously, the detachment element consists of one or several wires. The wires effectively lend themselves to transmit thrust or pull forces, which enables the attending physician not only to easily advance but also retract the implant. Ease of positioning is an important factor for the attending physician to be able to accurately move the implant to the desired placement site.

The single or multiple wires may have a round, square, oval or tubular cross section. Optional wire combinations may be employed in this case, for example combinations consisting of round and square wires. The wires are usually manufactured by drawing and thus reducing the raw material to the desired final cross section. The wire structure and thus the detachment characteristics of the detachment element can thus be influenced by the drawing process. By varying cross section and form of the wires these can be optimally adapted to suit the relevant application needs.

To simplify the detachment process further the detachment elements may be subjected to a surface treatment. For example, roughening the surface will result in accelerating the dissolution because this will cause the surface of the detachment element to be enlarged. There are other known possibilities to bring about a quicker dissolution of the detachment element by adopting an additional treatment method as has been described for instance in WO 03/017852 A1 mentioned hereinbefore to which reference is made in this respect. The detachment element may, for example, be subjected to a heat treatment process which causes the metal structure to be modified such that it very quickly disintegrates in an electrolyte when an electric voltage is applied. Such a heat treatment can be performed with the help of a laser, in a furnace or by means of an induction coil. Other types of pre-corrosion are also conceivable, for example by means of pre-etching. Moreover, detachment may also be accelerated passively by the designwise formation of local corrosion elements (e.g. magnesium-platinum elements). Such corrosion elements form when different noble metals are arranged next to each other, i.e. the detachment element is made of a metal less noble than the implant and/or the insertion aid.

Basically, the inventive device can be put to use in a different way in that the detachment or severance of the detachment element can be effected actively controlled from the outside or passively on its own. In the event of a passive dissolution the detachment element introduced into the blood vessel system dissolves automatically within a given time period without the need of additional steps being taken. Of course, this time span should be sufficiently long for the attending physician to be able to correctly position the implant and, if need be, make adjustments or again remove the implant from the catheter.

Preferred, however, is that dissolution is actively influenced in that the dissolution process in particular is effected by applying an electric voltage. The electric power may be alternate current or direct current, with a minor current intensity (<3 mA) being sufficient. In the event of direct current both polarities will basically be suitable.

To make it possible to actively influence or interfere with dissolution more effectively it may be expedient to design the detachment element in such a manner that formation of the above described local corrosion elements is avoided. This may be brought about e.g. by insulating the detachment element against adjacent areas of the device, for instance by means of insulating adhesive connections to be arranged between detachment element and implant.

The electrolytic severance is preferably achieved by making use of a power source to apply an electric voltage to the detachment element. In this case the detachment element for example acts as anode whereas the cathode is placed on the surface of the body. It is to be understood that the detachment element must be connected in an electrically conductive manner with the power source, in particular via the insertion aid.

Further steps may be carried out alternatively or additionally to support the dissolution or detachment of the detachment element. Mention shall be made in this context of the use of light waves, sound (ultrasound) or magnetic force.

It is, moreover, considered expedient to arrange for a securing means to be run through the implant. Securing means of this nature offer advantages by making the retraction of the implant into the catheter significantly safer which is necessary in case it has been wrongly positioned. Retracting, for example, an occlusion helix without securing means involves a risk in that portions of the helix are pulled apart and elongated due to the tensile or torsional stresses applied and in this way be plastically deformed irreversibly. In extreme cases the helix may separate or break giving rise to life-threatening embolism. Known from prior art are securing means made of flexible polymer threads or materials having shape-memory properties.

Publications also proposed the provision of devices comprising several detachment elements so that in case of need implants of variable length could be placed in position at the target location. In this manner it is possible, for instance, to place occlusion helixes of absolutely correct length in the aneurysm. Reference in this context is made to WO 01/32085 A1.

The application of such implants having a plurality of electrolytically corrodible locations is based on findings according to which the specific severance location of the implant situated nearest to the distal end of the catheter is dissolved by electrolysis when a current is applied to such a device. This is due to the fact that on the one hand the detachment locations in the catheter are isolated from the ionic medium through the catheter and thus not affected by electrolysis and, on the other hand, the current density decreases from proximal to distal owing to the distally increasing resistance. As a result of this, the electrolytically corrodible point which, viewed in distal direction, is closest to the distal end of the catheter is subjected to the most intensive electrolytic process and is thus preferentially dissolved.

The implants are in particular made of platinum or platinum alloys which have proven their worth. Such materials are also beneficial in that they have radiopaque properties and thus enable the placement of the implant to be easily monitored visually.

The device according to the invention may also be directly combined with a micro-catheter by means of which the implant is brought to its placement site. The catheter and the implant used in this case shall be matched with respect to their size. If necessary, the catheter also may exert constraint on the implant resulting in the implant to only assume a previously impressed secondary structure when it has been liberated and released from such constraint. Additionally, the catheter is moreover provided with radiopaque markers enabling a positioning in the target area with the help of known imaging methods.

The detachment element typically has a length ranging between 0.05 and 0.5 mm, in particular approx. 0.2 mm, and a diameter of between 0.05 and 0.5 mm, in particular of approx. 0.1 mm.

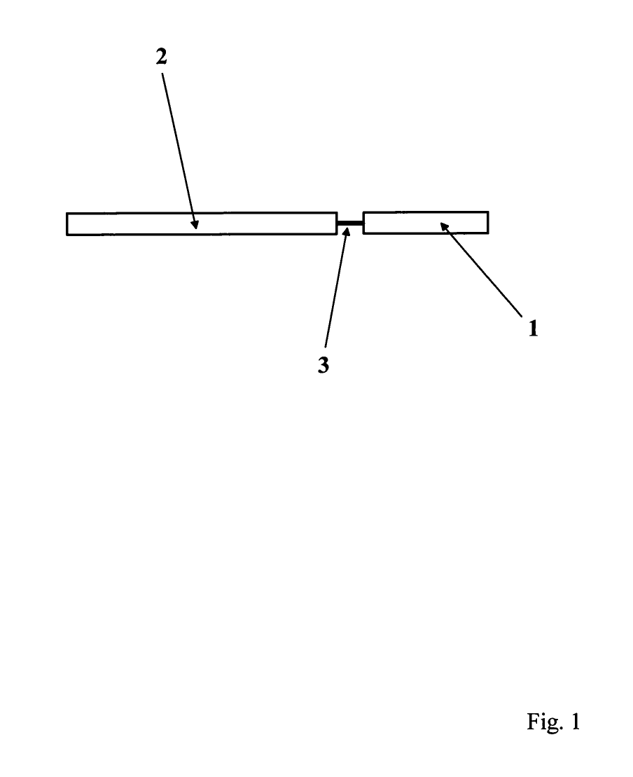

Further elucidation of the invention is provided through the enclosed FIG. 1 which illustrates the schematic design of the inventive device.

The device consists of an implant 1, an insertion aid 2 and a detachment element 3, wherein said insertion aid 2 is arranged at the proximal end of the device and the implant 1 at its distal end as viewed in the direction into which the device is advanced. The implant 1 is transferred to its placement site by moving the insertion aid 2 forward in a catheter not illustrated in the figure.

1. Device comprising an endovascular implant (1) to be introduced into blood vessels or body cavities of the human or animal body and an insertion aid (2), wherein the implant (1) and the insertion aid (2) being connected to each other via a detachment element (3) which is suitable to be dissolved at least partially after the device has been inserted into the body and in this manner causes the implant (1) to become detached or separated from the insertion aid (2), characterized in that the detachment element (3) is made of magnesium, a magnesium alloy and/or a biodegradable material.

2. Device according to claim 1, characterized in that the detachment element (3) is designed so as to be electrolytically corrodible so that a severance of the implant (1) is effected by applying an alternate current or direct current, preferably irrespective of the polarity.

3. Device according to claim 1, characterized in that after the device has been introduced into the body the detachment element (3) dissolves automatically within a certain time span to such an extent that the detachment of the implant (1) is effected.

4. Device according to claim 1, characterized in that the insertion aid is a guide wire (2).

5. Device according to claim 1, characterized in that the implant (1) is an occlusion helix, a stent or a retrieval system.

6. Device according to claim 1, characterized in that the detachment element (3) consists of one or several wires.

7. Device according to claim 6, characterized in that the wires have a round, square, oval and/or tubular cross section.

8. Device according to claim 1, characterized in that the detachment element (3) has a rough surface.

9. Device according to claim 1, characterized in that the detachment element (3) is pre-corroded.

10. Device according to claim 9, characterized in that the detachment element (3) is subjected to a heat treatment process.

11. Device according to claim 1, characterized in that said device is provided in combination with a micro-catheter.

12. Device according to claim 1, characterized in that the magnesium alloy in addition to magnesium also contains yttrium and/or rare earth metals.

13. Device according to claim 12, characterized in that the magnesium alloy contains up to 15% w/w, in particular up to 10% w/w of yttrium and/or rare earth metals.

14. Device according to claim 13, characterized in that the magnesium alloy contains 3.7 to 4.3% w/w of yttrium and 2.4 to 4.4% w/w of rare earth metals.

15. Device according to claim 1, characterized in that the biodegradable material is pure iron or an iron alloy with the exception of stainless steel.
